(19) United States
(12) Patent Application Publication
(10) Pub. No.: US 2023/0011166 A1
(43) Pub. Date: Jan. 12, 2023

(54) SYSTEM FOR PREDICTING TREATMENT OUTCOMES BASED UPON GENETIC AND PROTEOMIC IMPUTATION

(71) Applicant: Immunai, Inc., New York, NY (US)

(72) Inventors: Noam SOLOMON, New York, NY (US); Luis VOLOCH, New York, NY (US)

(21) Appl. No.: 17/679,034

(22) Filed: Feb. 23, 2022

Related U.S. Application Data
(63) Continuation-in-part of application No. 16/998,711, filed on Aug. 20, 2020, now Pat. No. 11,437,148.
(60) Provisional application No. 62/889,499, filed on Aug. 20, 2019.
(60) Provisional application No. 63/153,208, filed on Feb. 24, 2021.

(51) Int. Cl. G16B 20/00; G16H 10/60; G16B 40/20

(52) U.S. Cl. CPC G16B 20/00; G16B 40/20; G16H 10/60

(57) ABSTRACT

Methods, systems, and software provide machine learning and artificial intelligence including deep neural networks that enable the creation and operation of unique, AI-driven genomic and proteomic test results augmentation through variable genetic imputation.

### 1 COPYRIGHT NOTICE

A portion of the disclosure of this patent document may contain material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice shall apply to this document: Copyright Ⓒ 2019-2021, Immunai, Inc.

### 2 FIELD AND BACKGROUND OF THE TECHNOLOGY

### 2.1 Field of the Technology

The exemplary illustrative technology herein relates to systems, software, and methods for predicting gene and/or protein expression by applying an imputation algorithm to datasets comprising a range of genetic and/or proteomic markers. In certain example cases, such predictions might suggest treatment approaches such as the use of anti-inflammatory drugs if lots of pro-inflammatory cytokines are likely to be expressed. In some embodiments, the imputation algorithm can be used to ‘clean up’ dirty datasets - for example, scRNAseq data that is prone to ‘dropouts’ in instances where reverse transcription is not particularly robust. The technology has use in the fields of computing and medical diagnostic tools.

The use of artificial intelligence (AI) and deep neural networks (DNN) is transforming many fields including medicine; however, while linear or simple predictive models based upon mined medical records have become prevalent, their accuracy is generally low due to the lack of detailed information available about the patient’s specific disease condition and lack of ways to catch certain predictive patterns due to low input data quality.

Existing electronic health record (“EHR”) mining companies extract medical records from EHR data and provide structured medical information (diagnosis (DX), prescription/treatment (RX), and outcomes), but do not associate this information with known genetic sequences associated with a patient’s current conditions and do not correlate clinical outcomes with the patient’s collected genetic and proteomic information. For example, the EHR data is often incomplete and is often missing certain disease-associated genomic and proteomic data extracted from one or more test results included in the EHR to enable existing predictive systems to accurately make predictions, contributing to the poor outcomes from these existing systems. Examples of disease-associated genomic and proteomic data include tumor genomic data and/or protein analysis of proteins. , Embodiments for broader assays include patient genetic and proteomic data associated with a disease state.

In example embodiments, proteomic data may comprise a snapshot of a complete complement of proteins expressed in a biological sample at a specific moment in time. Assessing the proteome at any particular datapoint may involve three steps: (1) solubilizing and fractionating the proteome; (2) mass spectrometry analysis to measure the complement of proteins present in the sample; and (3) informatics analysis to analyze and assemble data into a snapshot of the proteome.

Existing genomic-sequencing companies provide sequencing services (e.g., DNA, RNA), and provide databases of known tumor types and their genomic sequences that can be interrogated in a variety of ways. However, these databases do not support predictive matching nor definitive matching on incomplete genomic data, nor do they provide recommended treatment (RX) and predicted outcome data.

In addition, these existing companies use common genomic sequencing techniques that frequently do not sequence enough of the available genomic information to enable a complete and accurate diagnosis and recommendation of the most effective therapy as the genomic information comprises one or more partially (either incomplete or low expression rate) expressed portions, or the available genomic sequence information does not permit identification of the most effective therapy. In some cases, the information needed to make an accurate determination has not been identified, in others the testing results are incomplete, or the sequencing technology has sensitivity limitations resulting in results using these test results that suggest several possible treatments and outcomes without differentiation as to which course of treatment may produce a better outcome for a particular patient. For example, in some cases, each single cell may not provide enough information to determine relative copy numbers. See e.g., Lähnemann et al, “Eleven grand challenges in single-cell data science” Genome Biology volume 21, Article number: 31 (2020), incorporated herein by reference. The ability to narrow the list of potential treatments and outcomes to the selected effective therapy (or therapies) is needed to prevent ineffective trials in the treatment regime; these trials increase costs and may decrease the survivability of the patients. Furthermore, the results of analysis of the medical records and treatment outcomes provides for earlier identification of potential success and failure of a current treatment regime.

For example, currently only about 30%-40% of patients treated with a specific immunotherapy see a partial or complete response to the treatment, which increases treatment timelines, allows disease progression, resulting in increased health care costs and exposes patients to unnecessary treatments and side effects, and reduces their overall quality of life. Similar limitations are present for many disease diagnosis/treatment pairs.

What is needed is a system that improves the accuracy with which a physician can select an immunotherapy treatment for a specific patient, in which the selected treatment is more likely than not to be effective, and according to which the patient outcomes can be monitored to quickly determine if the treatment is having a beneficial effect on disease progression or if an alternative treatment might be indicated instead.

### 3 SUMMARY OF THE EMBODIMENTS

The technology herein addresses the deficiencies and issues reviewed above by providing methods, systems, and software that enable the creation and operation of unique, AI-driven genetic test results augmentation via novel imputation techniques and provides an increased accuracy of prediction of patient treatment outcomes.

An embodiment of a system for variably imputing genetic and proteomic information into a collected data set may comprise storage access circuitry structured to read an input data set comprising genetic expression and proteomic data including genetic test results, and to read at least one imputation data set from a database, the imputation data set comprising one or more imputation technique definitions for variably imputing genetic and/or proteomic data; and an imputation engine coupled to a storage access circuitry, the imputation engine applying the one or more imputation technique definitions from the at least one imputation data set to a collected genetic and proteomic data set to variably impute genetic and/or proteomic data and create a resulting data set, wherein the storage access circuitry is further configured to output the resulting genetic dataset.

The imputation engine may select one or more imputation technique definitions from one or more trained models stored in the database and/or an imputation technique based in part upon at least one of a collected diagnosis, a collected treatment, a collected patient demographic, and/or a collected marker. In some embodiments, the collected marker is a genetic datum that is part of a collected data set.

The imputation engine may be further structured to provide an imputation technique by a using a selected trained model that produces changes in the collected genetic and proteomic data set to produce a changed genetic data set.

At least one imputation data set may be registered based at least in part upon a collected diagnosis, collected treatment, collected patient demographic, and/or a collected genetic datum present in the collected genetic and proteomic data set. The at least one imputation data set may comprise one or more imputation rules.

The resulting dataset may comprise one or more of the input genetic and proteomic data set(s), and a specified alteration to the input data set the imputation engine makes in response to execution of the one or more imputation rules and/or the input data set and added genetic and/or proteomic expression data and/or the input data set and at least one changed expression level of marker expression data and/or the input genetic and proteomic data set with at least one removed marker expression datum.

The one or more imputation rules may comprise an expression that is evaluated to determine if an imputation action specified by the one or more imputation rules is to be performed.

The imputation engine may be configured to evaluate the expression by an imputation rule execution function to determine a Boolean inclusion decision for an imputation rule. The Boolean inclusion decision may result in an imputation action specified in an imputation rule being executed by the imputation engine if the expression is determined to be true.

An embodiment of a method and/or computer readable media for variably imputing genetic and proteomic information into a collected data set may comprise reading an input genetic and/or proteomic data set comprising genetic expression data including genetic and/or proteomic test results, reading at least one imputation definition from a database, the imputation definition comprising one or more imputation technique definitions for variably imputing markers, applying the one or more imputation definition(s) to a collected data set to variably impute markers, and outputting the imputed markers as part of the variably imputed data.

The method and/or computer readable media may include selecting the one or more imputation definitions from one or more trained models stored in the database and/or selecting an imputation definition based in part upon a collected diagnosis and/or a collected marker in the collected data set and/or executing a selected imputation definition by executing a trained model that produces changes in the collected data set to produce a changed data set and/or selecting the at least one imputation data set based at least in part upon a collected diagnosis and/or collected marker data present in the collected data set.

The at least one imputation data set may comprise one or more imputation rules.

The outputted data may comprise at least one of the input data set and at least one specified alteration to the input data set in response to the execution of an imputation rule and/or the input data set and added marker data and/or the input genetic data set and at least one changed expression level of marker data and/or the input genetic data set with at least one removed marker datum.

The one or more imputation rules may comprise an expression that is evaluated to determine if a genetic imputation action specified by the one or more imputation rules is to be performed.

The method and/or computer readable media may further comprise evaluating the expression by an imputation rule execution function to determine a Boolean inclusion decision for the imputation rule. The Boolean inclusion decision may result in the imputation action specified in the imputation rule being executed if the expression evaluates as true.

An embodiment of a method of and/or a computer readable medium for predicting a treatment option or a disease outcome may comprise selecting, from a database comprising genetic data that may include sequence information, genetic and/or proteomic markers and other information, a set of genetic and/or proteomic markers corresponding to a disease or immune state; obtaining a set of genetic and proteomic markers by parsing a patient electronic health record (EHR); generating one or more imputed genetic and/or proteomic marker by applying one or more pre-determined rules to one or more of the obtained marker; generating a set of genome-specific and/or proteomic-specific trained model predicted outcomes by selecting from a set of specific trained models for each collected marker at least one specific trained model corresponding to one or more collected marker(s); using each of the selected specific trained model(s) to generate a genome-specific and/or proteomic-specific trained model predicted outcome, thereby generating a set of genome-specific and/or proteomic-specific trained model predicted outcomes; and using a second trained model to generate an overall outcome prediction using model inputs that comprise the set of genome-specific and/or proteomic-specific trained model predicted outcomes.

An embodiment of a method and/or computer readable medium of recommending a treatment or diagnostic test may comprise selecting, from a database of genetic and/or proteomic markers, a set of markers corresponding to a disease or immune state; obtaining a set of markers by parsing a patient electronic health record (EHR); generating one or more imputed markers associated with one or more of the collected markers; generating a set of genome-specific and/or proteomic-specific trained model predicted outcomes by selecting from a set of specific trained models at least one specific trained model corresponding to the collected marker; executing each of the selected specific trained model(s) to generate a specific trained \model predicted outcome, thereby generating a set of specific trained model predicted outcomes; process data with a second trained model to identify one or more specific trained model predicted disease states and/or outcomes, and recommending one or more identified treatments and/or diagnostic tests on the basis of the one or more predicted disease states and/or outcomes.

The method and/or computer readable medium may further include prescribing the recommended one or more identified treatments and/or diagnostic tests.

The recommending may include identifying one or more diagnostic tests and/or treatments for a predicted disease state.

The method and/or computer readable medium may further include using a selected trained model to identify genetic or proteomic imputations.

The method and/or computer readable media may further include selecting one or more rules, and evaluating an expression associated with each rule to identify genetic or proteomic imputations to be performed.

An embodiment of a predictor system for predicting a disease and/or treatment outcome may comprise at least one processor configured to perform operations comprising: selecting, from a database of markers, a set of markers corresponding to a disease or immune state; obtaining a set of markers by parsing a patient electronic health record (EHR); generating one or more imputed markers by applying one or more pre-determined rules to one or more of the collected markers; generating a set of genome-specific and/or proteomic-specific trained model predicted outcomes by selecting from a set of specific trained models at least one specific trained model corresponding to the collected marker(s); using machine learning based on each of the selected the trained model(s) to generate a specific trained model predicted outcome, thereby generating a set of specific trained model predicted outcomes; and using a second trained model to generate an overall outcome prediction using model inputs that comprise the set of specific trained model predicted outcomes.

An embodiment of a recommender system for recommending a treatment or diagnostic test may comprise at least one processor configured to perform: selecting, from a database of markers, a set of markers corresponding to a disease or immune state; obtaining a set of marker(s) by parsing a patient electronic health record (EHR); generating one or more imputed markers associated with one or more of the collected markers; generating a set of genome-specific trained model predicted outcomes by selecting from a set of specific trained models at least one specific trained model corresponding to the collected marker(s); executing each of the selected specific trained model(s) to generate a specific trained model predicted outcome, thereby generating a set of specific trained model predicted outcomes; executing a second trained model to identify specific trained model predicted disease state and/or outcomes; and recommending one or more of the identified treatments and/or tests on the basis of the predicted disease state and/or outcomes.

An output device may output a prescription for the recommended one or more identified treatments and/or tests.

The at least one processor may be further configured to identify one or more tests and/or treatments for a predicted disease state.

Machine learning hardware may use a selected trained model to identify genetic imputations.

The at least one processor may be further configured for selecting one or more rules, and evaluating an expression associated with each rule to identify genetic imputations.

An embodiment of a method and/or computer readable medium for improving the performance of a disease outcome prediction machine by generating input features for the disease outcome prediction machine comprising observed and imputed genetic and/or proteomic markers may comprise selecting a profile, from a database of disease profiles; mining a patient electronic health record (EHR) for observed markers that comprise one or more of the selected markers; and if one or more marker(s) that comprise the selected profile require modification, performing targeted imputation to produce imputed genetic marker(s); or if one of more markers that comprise the selected profile(s) are missing from both the observed markers and the imputed markers, generating one or more recommendations for deeper or targeted sequencing to create additional observed marker(s).

An embodiment of a disease outcome prediction machine for generating input features comprising observed and imputed markers may comprise: a selector circuit that selects, from a database of disease profiles, a data mining circuit that mines a patient electronic health record (EHR) for observed marker(s) that comprise the genetic markers of a profile, and at least one processor configured to: (a) perform targeted imputation to produce imputed marker(s) if one or more markers that comprise the disease profile are missing from the observed markers, and (b) generate one or more recommendations for deeper or targeted sequencing to generate additional observed marker(s) if one of more markers that comprise the disease profile are missing from both the observed and imputed marker(s).

An embodiment of a system for processing patient data to create imputation trained models may comprise a patient data collection engine configured to read and parse at least one electronic health record (EHR) dataset stored in a database comprising data from electronic health records (EHRs), the patient data collection engine comprising a patient EHR mining engine configured to: receive, from the database of EHRs, a plurality of patient EHRs, receive from a database of disease markers, a set of markers associated with at least one of the data elements in the received datasets; and mine the plurality of patient EHRs for observed disease marker(s), where the patient data collection engine is configured to interface with a hardware genetic material processor comprising a machine learning-based autoencoder configured to produce at least one trained model configured and trained to produce imputed markers based on observed markers, wherein each of the imputed markers corresponds to one or more of a received marker.

The autoencoder may comprise a neural network selected from the group consisting of an auto-encoder, a recurrent neural network, and a convolutional neural network. The autoencoder may be configured to create one or more rule-based encodings of the trained model.

The system may further comprise a database of methods for generating genetic and/or proteomic data, and a low abundance method recommendation engine configured to recommend one or more methods for generating genetic and/or proteomic data when markers are missing from both observed and imputed markers.

An embodiment of a method and/or computer readable medium for processing patient data to create imputation trained models may comprise providing a database of markers for imputation, providing a database of patient electronic health records (EHRs), operating a patient EHR mining engine to receive from the database of patient EHRs a plurality of patient EHRs, receiving from the database of markers a set of markers associated with a marker in the patient EHRs; and mining the plurality of patient EHRs for observed marker(s) associated with the received genetic marker information, using a machine learning-based deep neural network to produce at least one trained model configured and trained to produce imputed marker(s) based on observed marker(s) wherein each of the imputed marker(s) corresponds to a observed marker, and writing the at least one trained model to a data storage medium or device.

The neural network may be selected from the group consisting of an auto-encoder, a recurrent neural network, and a convolutional neural network. The method and/or computer readable medium may further include operating the auto-encoder to create one or more rule-based encodings of the trained model. The method and/or computer readable medium may further comprise providing a database of methods for generating genetic and/or proteomic data, and using a low abundance method recommendation engine to recommend one or more methods for generating additional data when low abundance or missing markers are identified in both the observed and imputed markers.

An embodiment of a system for imputing markers may comprise (1) a patient electronic health record (EHR) mining engine configured to receive from the database of EHRs a patient EHR, comprising marker data created using one or more particular assay arrays and mine the patient EHR for observed marker(s); (2) a long short-term memory (LSTM) autoencoder comprising at least one inner layer comprising a first set of weighted coefficients selected based on a set of markers that are not included in the one or more particular assay arrays, a second set of weighted coefficients selected based on polygenic risk scores associated with auto-immune pre-distribution, a third set of weighted coefficients selected based on polygenic risk scores associated with cancer pre-distribution, and a fourth set of weighted coefficients selected based on markers selected from a database of markers and statistics; wherein the LSTM autoencoder is configured to produce imputed markers based on at least one observed marker.

The system may comprise writing the imputed markers as a rule to a database.

The LSTM autoencoder may further comprise an input layer comprising markers identified by the particular assay arrays; and an output layer that provides marker(s) that include at least one imputed marker.

The set of markers that are not included in the one or more particular assay arrays may comprise a set of human leukocyte antigen (HLA) single nucleotide polymorphisms (SNPs); and the output layer provides the complete genomic sequence of the set of HLA-A and HLA-B genes and/or the output layer may provide a complete genome and/or protein description.

The LSTM autoencoder may be trained with data selected from: (1) markers identified by one or more particular assay arrays; (2) markers that are not included in the one or more particular assay arrays; (3) polygenic risk scores associated with at least one of autoimmune and cancer pre-distribution; and (4) markers selected from a database of markers and statistics.

The LSTM autoencoder may be trained with data selected from markers identified by one or more particular assay arrays and/or data selected from markers that are not included in the one or more particular assay arrays and/or data selected from polygenic risk scores associated with at least one of auto-immune and cancer pre-distribution and/or data selected from markers selected from a database of markers and statistics.

An embodiment of a method and/or computer readable medium for imputing marker(s) may comprise receiving from the database of electronic health records (EHRs) a patient EHR comprising marker data created using one or more particular assay arrays; mining the patient EHR dataset for observed marker(s); and operating a long short-term memory (LSTM) autoencoder comprising at least one inner layer comprising a first set of weighted coefficients selected based on a set of markers that are not included in the one or more particular assay arrays, a second set of weighted coefficients selected based on polygenic risk scores associated with auto-immune pre-distribution, a third set of weighted coefficients selected based on polygenic risk scores associated with cancer pre-distribution, and a fourth set of weighted coefficients selected based on markers selected from a database of markers and statistics; wherein the LSTM autoencoder produces imputed markers based on the observed marker(s).

The method and/or computer readable medium may include writing the imputed markers as a rule to a database.

The set of markers that are not included in the one or more particular assay arrays may comprise a set of human leukocyte antigen (HLA) single nucleotide polymorphisms (SNPs); and the output layer provides the complete genomic sequence of the set of HLA-A and HLA-B genes.

The set of markers that are not included in the one or more particular assay arrays may comprise a set of human leukocyte antigen (HLA) single nucleotide polymorphisms (SNPs); and the output layer provides a complete genome.

The method and/or computer readable medium may further comprise training the LSTM autoencoder with data selected from: markers identified by one or more particular assay arrays; markers that are not included in the one or more particular assay arrays; polygenic risk scores associated with at least one of auto-immune and cancer pre-distribution; and markers selected from a database of markers and statistics.

The method and/or computer readable medium may further comprise training the LSTM autoencoder with data selected from markers identified by one or more particular assay arrays.

The method and/or computer readable medium may further include training the LSTM autoencoder with data selected from markers that are not included in the one or more particular assay arrays and/or data selected from polygenic risk scores associated with at least one of auto-immune and cancer pre-distribution and/or markers selected from a database of markers and statistics.

An embodiment of a method and/or computer readable medium for generating imputation trained models may comprise collecting a set of collected marker(s) from a training database, imputing missing or low expressed marker(s), creating processed collected data, and creating a trained model from the processed collected data.

Creating the one or more imputation trained models may include training an LSTM autoencoder with a training dataset comprising: markers identified by one or more particular assay arrays; markers that are not included in the one or more particular assay arrays; polygenic risk scores associated with at least one of auto-immune and cancer pre-distribution; and markers selected from a database of markers and statistics.

The trained LSTM auto encoder may comprise an input layer comprising SNPs identified by particular assay arrays, an inner layer comprising selected weights (loss functions) comprising a set of SNPs (e.g., HLA SNPs) that are not part of the selected assay arrays, and weights associated with each SNP; an inner layer comprising selected weights (loss functions) comprising selected weights (loss functions) comprising parameters from polygenic risk scores associated with auto-immune diseases; an inner layer comprising selected weights (loss functions) comprising parameters from polygenic risk scores associated with cancer pre-disposition; an inner layer comprising selected weights (loss functions) comprising parameters identified in the literature (e.g., Stanford GWAS, literature searches); and an output layer that provides the complete genome of HLA-A and HLA-B genes.

Training the one or more imputation trained models may include generating, with the trained LSTM autoencoder, one or more entries in an imputation possibilities database where each entry includes a rule for generating at least one imputed marker based on at least one observed marker; and training an expert rules module to produce an imputed marker using a rule and at least one observed marker.

An embodiment of a system for generating imputation trained models may comprise at least one processor configured to collect a set of collected marker(s) from a training database, at least one neural network configured to impute missing or low expressed marker(s), creating processed collected data, and a machine learning device configured to create a trained model from the processed collected data.

The machine learning device may include an LSTM autoencoder with a training dataset comprising: markers identified by one or more particular assay arrays; markers that are not included in the one or more particular assay arrays; polygenic risk scores associated with at least one of auto-immune and cancer pre-distribution; and markers selected from a database of genetic markers and statistics.

The trained LSTM auto encoder may comprise an input layer comprising SNPs identified by particular assay arrays, an inner layer comprising selected weights (loss functions) comprising a set of SNPs (e.g., HLA SNPs) that are not part of the selected assay arrays, and weights associated with each SNP; an inner layer comprising selected weights (loss functions) comprising parameters from polygenic risk scores associated with at least one auto-immune disease; an inner layer comprising selected weights (loss functions) comprising parameters from polygenic risk scores associated with cancer pre-disposition; an inner layer comprising selected weights (loss functions) comprising parameters identified in the literature (e.g., Stanford GWAS, literature searches); and an output layer that provides the complete genomic sequence of HLA-A and HLA-B genes.

The machine learning device may include a trained LSTM autoencoder configured to generate one or more entries in an imputation possibilities database where each entry includes a rule for generating at least one imputed marker(s) based on at least one observed marker; and a trained expert rules module trained to produce at least one imputed marker using a rule and at least one observed marker.

An embodiment of a system for imputing marker(s) may comprise a database of rules for generating imputed marker(s) based on information that includes observed markers; and a rules engine for selecting one or more rules from the database of rules and for using the selected one or more rules to produce imputed marker.

An embodiment of a system for improving the performance of a disease outcome prediction machine by generating input features for the disease outcome prediction machine comprising observed and imputed markers may comprise a storage access circuit configured to select, from a database of disease profiles, a patient database access processor configured to mine a patient electronic health record (EHR) for observed marker that comprise selected markers, and an imputation engine coupled to the patient database access processor and the storage access circuit, the imputation engine being configured to perform operations comprising if one or more markers that comprise the selected disease profile are missing from the observed markers, performing targeted imputation to produce imputed marker(s), and if one of more markers that comprise the selected disease profile are missing from both the observed and imputed marker(s), generating one or more recommendations for deeper or targeted sequencing to produce additional observed marker.

An embodiment of a method and/or computer readable medium for imputing markersmay comprise collecting a set of collected marker(s), generating a set of missing markers that includes selected markers that are not included in a set of collected markers, including selecting the marker(s) from a database of markers associated with a disease state; and imputing at least one marker for one or more of the markers in the set of missing markers.

Collecting markers may include parsing a patient electronic health record (EHR) to select a set of collected marker and/or reading genetic and/or proteomic marker information from a testing device.

Imputing a marker may include selecting from among a set of imputation trained models a particular imputation trained model that has been trained to impute only the selected set of markers; and executing the particular imputation trained model to produce at least one imputed marker of the selected set of markers.

Imputing a marker may include selecting from an imputation possibilities database one or more imputation rules wherein each imputation rule corresponds to one or more collected markers and to one or more markers; and executing with a trained imputation model the one or more imputation rules to produce one or more imputed marker.

Executing the one or more imputation rules may include generating an imputed strength of expression associated with one or more imputed marker.

An embodiment of a method and/or computer readable medium for generating imputed markers for inclusion in a processed collected dataset corresponding to a disease or immune state may comprise creating one or more imputation trained models to produce one or more imputed markers wherein the one or more markers comprise markers that are associated with a specific disease or immune state; collecting observed disease/immune state markers from a collected dataset; imputing markers related to the disease/immune state and the collected data, and including the produced imputed data into a processed collected dataset.

The imputing may further comprise selecting one or more imputation trained models for use, and executing one or more of the imputation trained models to produce imputed marker(s) corresponding to missing markers in the observed markers and/or identifying a database of imputation rules associated with the identified disease/immune state, selecting one or more imputation rules to evaluate on the basis of the identified disease/immune state, and evaluating the selected rules, and if a rule evaluation evaluates as TRUE, implementing the rule action to effect the imputation.

An embodiment of a system for imputing marker(s) may comprise at least one processor configured to collect a set of collected marker(s), the processor further configured to produce a set of missing markers that includes selected markers that are not included in a set of collected markers, and to select the markers from a database of markers associated with a disease state; and an imputation engine configured to impute at least one marker for one or more of the markers in the set of missing markers.

The processor may be configured to parse a patient electronic health record (EHR) dataset to select a set of collected marker(s), and/or to read genetic information from a genetic testing device, and/or read proteomic information from a testing device that is capable of processing proteomics.

The imputation engine may comprise a selecting circuit configured to select a particular imputation model that has been trained to impute only the selected set of markers from among a set of imputation trained models; and an execution circuit that executes the particular imputation model to produce the imputed marker and/or an imputation possibilities database that provides one or more imputation rules wherein each imputation rule corresponds to one or more collected markers and to one or more imputable markers; and a trained imputation model that executes the one or more imputation rules to produce one or more imputed marker(s).

The trained imputation model produces an imputed strength or other quality or characteristic of expression associated with one or more imputed marker(s).

An embodiment of a system for generating imputed markers for inclusion in a processed collected dataset corresponding to a disease or immune state may comprise one or more imputation trained models configured to produce one or more imputed markers, comprising markers that are associated with the disease or immune state; a collected dataset providing collected observed disease/immune state specific markers; an imputation engine that imputes markers related to the disease/immune state and the collected data; and an output device that incorporates the produced imputed data into a processed collected dataset.

The imputation engine may further comprise a selector that selects one or more imputation trained models for use and an execution core that executes one or more of the imputation trained model(s) to produce one or more imputed marker(s) corresponding to any markers missing from the set of observed markers.

The imputation engine may comprise a database of imputation rules associated with markers of an identified disease/immune state, a selector that selects one or more imputation rules to evaluation on the basis of the identified disease/immune state, and a processor that evaluates the selected rules, and in response to such evaluation (e.g., if a rule evaluation evaluates as TRUE), implements the rule action to effect the imputation.

The above techniques may be used individually or in combination. For example, any of the following techniques and associated structures and systems that implement the techniques (including embodiments of systems, methods and/or computer readable media that implement same) may be used together in any combination or subcombination:
(a) variably imputing genetic and/or proteomic information into a collected data set,
(b) predicting a disease and/or treatment outcome,
(c) recommending a treatment or diagnostic test,
(d) a recommender system for recommending a treatment or diagnostic test,
(e) improving the performance of a disease outcome prediction machine by generating input features for the disease and/or treatment outcome prediction machine,
(f) a disease outcome prediction machine for generating input features comprising observed and imputed markers,
(g) processing patient data to create imputation trained models,
(h) imputing marker(s),
(i) generating imputation trained models,
(j) improving the performance of a disease outcome prediction machine by generating input features for the disease outcome prediction machine comprising observed and imputed markers,
(k) generating imputed markers for inclusion in a processed collected dataset corresponding to a disease or immune state,
(l) generating imputed markers for inclusion in a processed collected dataset corresponding to a disease or immune state.

These and other aspects and advantages will become apparent when the Description below is read in conjunction with the accompanying Drawings.

### 4 BRIEF DESCRIPTION OF THE DRAWINGS

The features of the present technology will best be understood from a detailed description of the technology and example embodiments thereof selected for the purposes of illustration and shown in the accompanying drawings.

FIG. 1 illustrates an exemplary system architecture/data flow for training and diagnostic use of the system as implemented by the current technology.

FIG. 2 illustrates an exemplary computer implementation of the training portion of the current technology.

FIG. 3 illustrates an exemplary computer implementation of a neural network coupled to a coeffcient database.

FIG. 4 illustrates an exemplary computer implementation of the training portion of the current technology.

FIG. 5 illustrates an exemplary process flow for the training portion of the current technology.

FIG. 6 illustrates an exemplary process flow for the diagnostic use of the current technology.

### 5 DESCRIPTION OF SOME EMBODIMENTS OF THE TECHNOLOGY

### 5.1 High Level Overview

The technologies support accurate recommendation of specific therapies, and the prediction of patient health outcomes in spite of the wide variation in patient immune system responses and known immunotherapy outcomes by using a collection of machine learning techniques for diagnosis (DX)/treatment (RX) and outcome prediction, utilizing a combination of selected genetic imputation and trained expert systems, and ongoing monitoring of patient progress to determine the correlation between predicted and actual patient outcomes.

The exemplary technologies presented herein use artificial intelligence (AI) and deep neural network (DNN)-based techniques as applied to complex datasets comprising the combination of medical data obtained from EHR mining, collection of detailed proteomic and genetic data further comprising one or more of DNA tumor sequences, a subset of the circulating free DNA (cfDNA) obtained from blood and/or plasma samples comprising DNA shed from tumor cells, tumor imaging, genetic data derived from cells of the immune system, genetic sequencing of microbiome, protein information obtained from protein and proteomic assays, and data from other similar assays, the set of which is enhanced using a novel imputation methodology that increases the robustness of the input data set for a particular patient using imputation strategies and methods that are less resource intensive than existing methods. The exemplary technologies presented herein improve the results obtained using a trained deep neural network for predicting disease progression and outcomes over existing imputation methods without incurring the increased computing resource costs required by existing imputation methods.

The ongoing monitoring techniques permit the system to determine how an individual is responding to a specific therapy and enables the prediction to be refined on the basis of observed response and any additional test results that may become available.

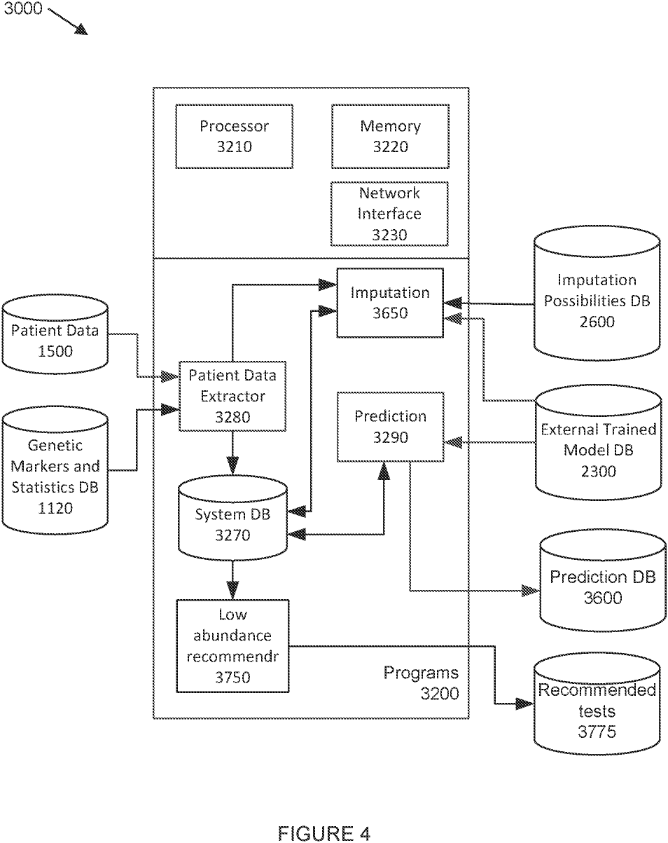

The exemplary technologies described herein extend EHR mining results to include complete outcome information for historic cases (length of survival, disease progression timelines), as well as enabling the ongoing collection and inclusion of genetic and/or proteomic data that may not have been available in early stages of treatment. The EHR mining component further supports the inclusion of patient-specific markers (e.g., genetic and proteomic markers, immune-specific markers, microbiome markers), which are used by the current technology as part of its data completion (e.g., imputation), predictive, and recommender features. When the system determines that conditions exist where insufficient information is provided in the mined EHR dataset that cannot be resolved by the imputation features of the methods described herein, the system recommends further activities (e.g., tests, procedures) capable of generating additional information in order to improve the predictive and/or recommender outcomes of the system (e.g., additional or deeper sequencing). These recommended tests and procedures are identified in a database (e.g., recommended tests database, FIG. 4, 3775). Insufficient information includes conditions that produce imputation, predictive, or recommendations with low confidence, or confidence below a defined threshold stored in a system configuration. The identified tests may be used to perform additional testing on previously collected and retained biological samples or may include collecting new samples from a patient. For example, the system may determine that it is unable to produce a recommended therapy with a confidence above a preset threshold of 75%, and thus generate a recommendation for additional sequencing tests (as described below).

Because common genetic sequencing and proteomic techniques, especially those used in single cell analysis workflows, often do not identify complete enough information (e.g., they fail to sequence enough of the patient’s genetic information to enable identification of an effective therapy, or contain “dropouts” where expressed genes may be missed because reverse transcription is not robust), taking into account all available information, or the testing that has been performed to date does not identify a sufficiently robust list of proteins and/or a sequence or set of sequences that enable the identification of an effective therapy, the described technology uses a data completion model for genetic imputation to enhance the collected data in order to improve the technologies’ predictive outcomes. This reduces the number of potential treatments considered by more completely associating the state of the collected data, the treatments performed, and known outcomes in other cases and permits the system to more accurately identify likely effective treatments along with their predicted outcomes. The ability to narrow the list of potential treatments and outcomes to one or more likely effective therapies associated with specific outcomes reduces the administration of ineffective and costly immunotherapies or other treatments as part of the treatment regime.

Lastly, the described technology provides for ongoing monitoring of the patient’s progress on a specific immunotherapy, which enables the treating physician to more quickly determine that a patient’s tumor is not responding to that therapy, leading to earlier termination of ineffective treatment regimens and modification of the treatment plan. Existing prediction systems do not track a patient’s progress over time, are not capable of providing early indications that a treatment is not working and that an alternative treatment should be considered. The training, prediction, and tracking capabilities of these types of systems may be improved to > 62% accuracy or > 60% accuracy or > 50% accuracy or > 40% accuracy or > 30% accuracy or > 25% accuracy by using the techniques and technologies described herein by highlighting a large fraction of patients that are at risk of non-response.

Thus, the described system facilitates improved selection of effective immunotherapy treatment options for each individual patient, in which the selected treatment is more likely to be effective, and monitors patient outcomes in order to quickly determine if the treatment is having the expected effect or if an alternative treatment would be more effective.

The accuracy of the machine learning and prediction/recommendation process steps are optionally improved by pre-processing collected data to impute missing, incomplete, and/or low expressed genetic information in the collected dataset, creating a pre-processed collected dataset. This allows the machine learning models to more accurately predict outcomes and recommend treatments by eliminating conditions in which there is insufficient distinguishing data in the patient’s collected data. The imputation process imputes genetic and/or proteomic data using one or more imputation trained models configured and selected to determine missing, incomplete, and/or low expressed data. In this way, each trained model is an imputation definition for a specific condition. In some embodiments, a general imputation may be performed where all imputable genetic and proteomic information is imputed by the system. In some embodiments, a specific imputation may be performed, such a specific imputation operation that imputes (e.g., genetic and/or proteomic) information associated with one or more particular disease conditions. The specific imputation alternative embodiment allows the prediction model to save computing resources by not imputing information that is not needed for prediction and/or recommendation activities. Limiting the process to imputing only missing information associated with one or more specific diseases further increases the resolution and accuracy when imputing low abundance genetic and/or proteomic data that may be missing in the collected data. The prediction process further imputes missing data associated with one or more identified diseases, and generates a prediction value representing confidence in, or strength of, imputation, using one or more imputation possibility rules. These rules are generated during model training (using training datasets) as described below. This allows the system to use a low-resource rules-matching imputation method rather than employing a known resource-intensive imputation method when processing collected data.

The described system includes the hardware and software components necessary for processing a variety of clinical data and predicting patient outcomes for immunotherapeutic treatment of disease, comprising at least one trained predictive model which in an exemplary embodiment is a comprising a deep neural network of at least 6 layers, in which the predictive model is trained on a combination of patient EHR data and/or directly read genetic and/or proteomic data and predicts at least one of the following: a disease diagnosis, the effectiveness of a disease treatment, a disease outcome, a disease progression over time, or disease survival.

In more specific embodiments, the described system and methods predict outcomes of therapies on disease and/or immune states treatable with specific therapies including predicted outcomes of these specific therapies. In these more specific embodiments, the described system and methods comprise collecting incomplete patient data, imputing missing data on the basis of the collected genetic and proteomic data to produce processed collected genetic and proteomic data, and using the processed collected genetic and proteomic data with the trained predictive model in order to determine one or more of a disease diagnosis, the effectiveness of a disease treatment, a disease outcome, a disease progression over time, or disease survival.

These and other aspects and advantages will become apparent when the Description below is read in conjunction with the accompanying Drawings.

### 5.2 Definitions

The following definitions are used throughout, unless specifically indicated otherwise:

| | |
|---|---|
| TERM | DEFINITION |
| Genetic data | Germline sequence data, mutation data, single cell RNA sequencing (scRNAseq), microbiome sequencing, metabolomic data, transcriptomic, epigenomic, DNA fragmentomic, and other genetic and genomic test results. |
| Proteomic data | Data related to identification and quantification of a specific identified protein or the complete protein complement of a cell or tissue. |
| GP data | Genetic data and/or Proteomic data |
| Collected dataset | Genetic and/or proteomic data extracted from one or more EHR record (s) and/or collected experimentally. |
| Produce | Create, modify, or remove a genetic or proteomic marker under the control of the imputation process. |
| | Producing a dataset includes creating a dataset including at least one produced genetic or proteomic marker. |
| EHR | Electronic Health Record Comprises medical records including diagnosis (DX) and treatment (RX) information and standard medical laboratory test results, as well as proteomic, genetic and genomic test results that comprise genetic or proteomic data. |
| Marker | A genetic or proteomic data point, comprising an identifier (ID or tag) identifying the datum, an optional value, and an optional expression level. Operations on a marker may be upon the marker as a whole, or upon an individual element of the marker. |
| | A genetic marker is a marker that represents genetic data. |
| | A proteomic marker is a marker that represents protein / proteomic data. |
| | A Marker may comprise or include a data structure. |
| Disease profile | A set comprising one or more genetic and/or proteomic markers, typically associated with a disease and/or immunologic state. |
| SNP | Single Nucleotide Polymorphism occurs where there is a substitution of a single nucleotide at a specific position in the genome, and the substitution is present in 1% or more of the population. Some SNPs are predictors of susceptibility to some genetic-based diseases such as sickle-cell anemia and cystic fibrosis, and also predictors of how the body will respond to treatment. |
| VCF | Variant Call Format, a standardized text file format for representing SNP and other genetic information. |
| Microbiome | The combined genetic material of the micro-organisms present in a particular physiological environment such as, for example, the gut or the skin. |
| Disease areas | Auto-immunity, oncology (e.g., cancers (general), breast cancer, hematologic malignancies) |
| Diagnosis (DX) | The identification of a disease or illness, sometimes associated with a well-known identifier such as an ICD code. |
| Treatment (RX) | Medical care provided to a patient for an illness or injury, sometimes associated with a well-known identifier such as an ICD code. |
| | Treatments may include, for example, immunotherapy, chemotherapy, radiation, anti-inflammatory drugs. |
| Outcome | The effect of a treatment on a patient’s health and/or disease course. |
| Imputation engine | The program or programs that perform a variable imputation process |

### 5.3 Detailed Description of Exemplary Embodiments

### 5.3.1 Data Flow for Exemplary Training Mode Operation

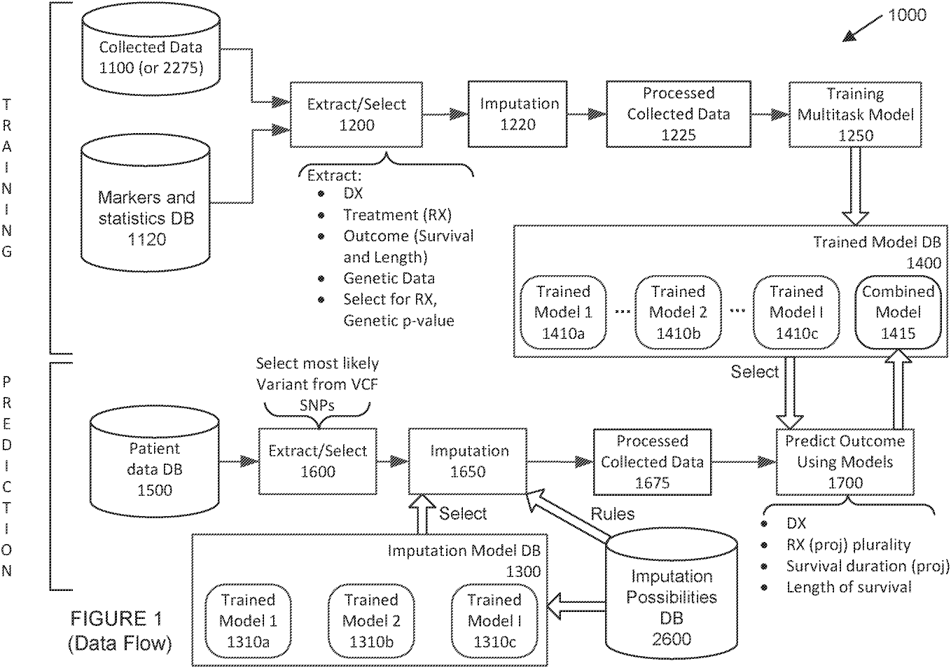

FIG. 1 illustrates an exemplary data flow and interaction (1000) between the training and prediction systems and provides a general example of how the system operates to produce improved results.

Machine learning techniques are typically ineffective, or at best partially effective, in analyzing patient EHR data for determining the best outcome for patients with specific cancer types, treatments, and outcomes because the training datasets often do not have sufficient genetic and/or proteomic information in the encoded patient EHR data to establish necessary correlation(s). Thus, the models produced by training a machine learning system on this type of data do not accurately predict treatment/outcome for a specific patient with a definitive diagnosis and supporting lab work. Improvements can be made to the data flows in the model training processes and the patient EHR processing in order to produce a system that can accurately predict the outcome of various treatment regimes.

The training process improvements include the combination of parsing EHR records to generate a training dataset storage database (2275) along with statistical significance data that indicates significance of the information that may be found in the parsed EHR data. Exemplary statistical significance data includes p-values that indicate the relevance of particular markers, and/or relevance of traits associated with the markers, to particular diseases or conditions. The statistical significance data are taken from a database of markers and their associated statistics (e.g., database 1120). For example, the genomic database may be generated by extracting summary statistics, including p-values, from Genome Wide Association Studies (GWASs), both public and private, and in particular, from GWASs that are related to specific disease pathologies, such as oncology and immunology. GWAS identifies inherited genetic variants associated with a risk of disease or a particular genetic trait. Other databases may be used as training sources for protein and proteomic data as understood by those skilled in the art.

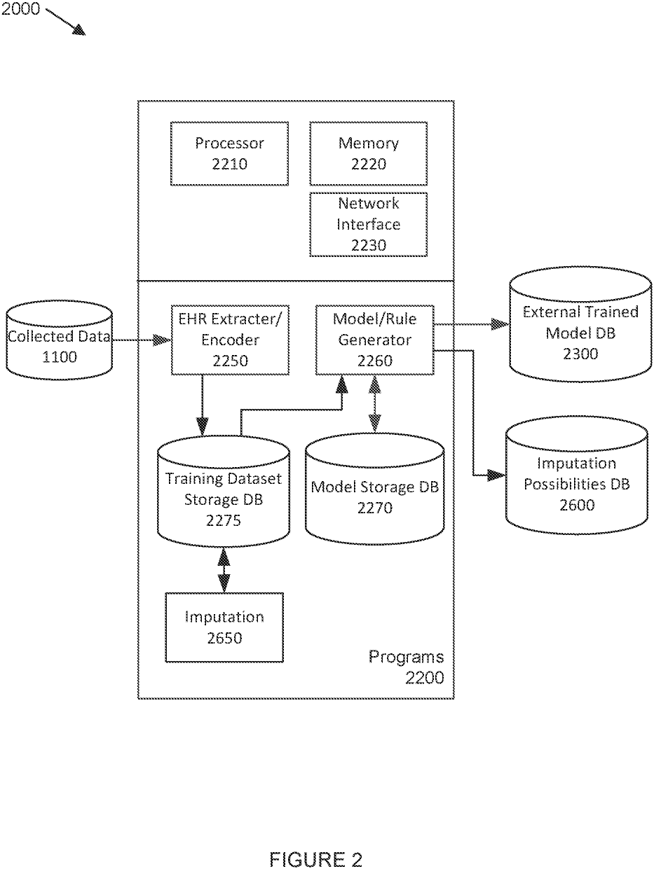

The EHR data is mined (1200) for diagnosis (DX), treatment (RX), and outcome (e.g. partial response, complete response, progression-free survival, length of survival) information, and any additional data needed to create at least one collected dataset. The statistical significance data is used to select markers from the collected dataset for inclusion in training datasets based upon the usefulness of the markers for determining diagnosis, treatment, and predicted outcomes, as assessed in part by their p-values. Genetic and proteomic data is optionally imputed by the training dataset imputation process (1220) with the collected dataset(s) to generate one or more processed collected dataset(s). The processed collected dataset(s) comprise collected and imputed genetic and/or proteomic data that includes a more complete set of the selected markers than the collected dataset(s) contains. The imputation process may be performed across all of the collected dataset(s), or may be performed limited on the basis of one or more of diagnosis and/or treatments identified in a collected dataset(s). The training dataset imputation process (1220) operates in a manner consistent with the steps described below for the imputation process (1650), and uses either (or both) trained models from the imputation model database (1300) and rules from the imputation possibilities database (2600). If imputation is not performed, the collected dataset is copied to the processed collected dataset. The processed collected dataset is stored in the training dataset storage database (FIG. 2, 2275).

The resulting collected dataset and/or one or more processed collected datasets are used to fully train (1250) one or more multi-task trained prediction models. The resulting trained models (e.g., models 1410a, 1410b, ...) are stored in a trained model database (1400), indexed by one or more of diagnosis, treatment, and marker. The trained models are used by the system to complete datasets by imputation (e.g., an imputation trained model), recommend treatment courses, and predict treatment outcomes.

Additional training process improvements involve producing one or more imputation trained models that identify genetic and/or proteomic information to be imputed in a patient’s collected dataset when incomplete input information is discovered. In some embodiments, the training system uses one or more disease profiles to determine a set of associated genetic and/or proteomic information. The profiles may be implemented as associations between specific genetic and/or proteomic information identified in a database. The training system uses a model/rule generation program (FIG. 2, 2260) to generate entries in an imputation possibilities database (2600) that includes one or more rule definitions identifying mapping of strengths of measured markers to imputed markers. The training system model/rule generator (2260) also trains imputation models to determine one or more missing markers in a patient collected dataset based on other information present in the patient collected dataset and training collected datasets (e.g., RX, DX, patient demographic data), in some embodiments based on information in the imputation possibilities database, and stores the resulting imputation trained models (e.g., trained model 1310a, 1310b, ...) in an imputation model database (1300). The imputation trained models provide an imputation definition and are subsequently used to impute least one aspect of an individual patient’s markers in a patient’s collected dataset.

### 5.3.2 Data Flow for Exemplary Predictive Mode Operation

After the trained model databases are constructed, the system operates in predictive mode by accepting patient-specific genetic and EHR data and replicating the previously described data mining operations to identify DX, RX, outcome (to date), and genetic and/or proteomic data (e.g., tumor, SNP, VCF, and microbiome) associated with the patient and writes the data to the patient database (1500). This step is performed by the patient dataset extractors program (FIG. 4, 3280). The information collected is generally incomplete, as the patient is undergoing treatment or is being monitored post-treatment. The information associated with a particular patient is read from the patient collected dataset database (1500) and/or one or more EHR programs (not shown). These sources comprise one or more collected datasets and/or processed collected datasets associated with specific patients. Alternatively, some of the information may be read directly from assay devices, for example, genetic sequencing equipment and assay array readers (such as an Affymetrix GeneChip scanner not shown) for collecting genetic data, similar devices and systems for protein and proteomic assays, and/or from other sources.

The patient’s collected dataset(s) is then ordered on the basis of genetic data corresponding to one or more diagnoses or treatments. In one exemplary embodiment, the collected information is ordered by the most likely variant using the VCF SNP information (1600) (which is obtained from summary statistics, GWAS-like work, or other predictive models of immune and oncology-related traits) and the system proceeds to the imputation step (1650). The imputation step is provided by a genetic and proteomic imputation program selecting and executing on a computer (e.g., imputation program FIG. 2, 2650 or FIG. 4 , 3650). In some embodiments, the imputation program is shared across multiple imputation process sets and is called an imputation engine.

The imputation engine operates on the genetic and/or proteomic information from one or more datasets (either patient’s collected data or training collected data) in order to enhance the dataset(s) by imputing missing data using one or more imputation trained models (taken from the above-mentioned imputation database 1300). Alternatively or additionally, in some exemplary embodiments, rules stored in imputation possibilities database (2600) are used during imputation.

The process of imputing data completes missing (or low expressed) portions of the patient’s genetic and/or proteomic information (in certain single cell sequencing embodiments, and depending on the assay, certain types of results may be either obtaining the sequence or not). In some embodiments, the system limits imputation to specific markers on the basis of information in the dataset, e.g., patient data such as RX, DX, demographic data, or specific markers that are included in the database of markers (which may by additionally identified as associated with specific type of patient data), markers having a significance greater than a threshold value, or markers that are associated with an identified disease, treatment, tumor, or immunological state. In an exemplary embodiment, the system limits imputation to missing markers that have an established association with a disease identified in the patient’s dataset. Alternatively, the limitation may be for imputations where the p-value associated with a particular marker is greater than a pre-determined and stored threshold value, for example, a threshold value greater than 75%, greater than 80%, or greater than 90%. The threshold value is associated with each set of selection parameters (such as RX, DX, specific demographic data) and is stored in a system configuration or database (not shown). The variable nature of the imputation process allows prediction models to be more accurate in their predictions by eliminating conditions in which insufficient distinguishing data is in the patient’s records, by being able to utilize a wider set of features that have known association with the patient’s particular condition, as well as in aggregating data sources from different datasets. Note that the imputation process varies depending upon the set of selection parameters, we call this selective imputation as variable imputation. The processed collected data (1675) is optionally logically associated with the patient’s EHR data, and is passed, along with the patient’s EHR data, to the prediction step.

The prediction step (1700) uses the trained prediction models from the prediction model database (1400) against the patient’s processed collected dataset (1675) in order to establish or confirm a diagnosis, identify and recommend one or more treatments (RX) in ranked order of predicted effectiveness given the patient’s processed collected data, and predict the outcomes of each treatment (survival, length, and end state).

The prediction step comprises selecting and executing a set of specific trained models (1410a, 1410b, 1410c) each of which is trained to predict the effects of a particular marker in the input set of measured and imputed markers. For example, with genomic data, each specific trained model predicts the outcome influenced by the particular gene in light of the DX and RX (e.g., the diagnosis and drug response). In some alternative embodiments, the prediction step uses specific trained models configured for proteomic markers, or may use specific trained models configured for genomic and proteomic markers. Collectively, the genomic-specific and proteomic-specific trained models are called specific trained models.

The predicted outcomes generated by one or more specific trained models are optionally combined to create a combined trained model (1415).

The combined trained model is created by training the model to predict the results of the combination of outcomes predicted for each of the markers represented by the individual specific trained models. Based on a combination of outcomes predicted by multiple specific trained models, the combined trained model generates a single trained model output that predicts one or more of the following: a disease diagnosis (DX), a treatment recommendation (RX), patient outcomes, and probabilities for each outcome. An exemplary combined trained model output includes one or more predicted outcomes based upon the combination of the determined genetic, proteomic, DX, and RX information. Specifically, in some exemplary embodiments, the resulting combined trained model output includes prediction of a drug (RX) response based upon one or more markers identified as present in the collected and processed collected datasets. Exemplary combined trained model output includes one or more predicted outcomes, each with an associated outcome probability, and each based on a different treatment (RX) recommendation.

The two-step prediction method of the technology described herein has a number of advantages over known prediction systems that may use a monolithic trained model to predict outcomes. For example, by selecting and executing first specific trained models specific to certain markers, the system saves computing resources by only reasoning over a disease-specific subset of all known markers using models that each include many less nodes or other model parameters than would be required for a monolithic model trained on a larger set of markers. Further, the arrangement of multiple specific trained models can be executed using parallel processing techniques, as are known to those having skill in the art, which may save time required to generate an outcome. In addition, training and prediction efficiency of the second model is improved by providing second model inputs that comprise information that has been reasoned over by the first, specific, trained models rather than raw or unprocessed input data. In this manner, an exemplary embodiment includes multiple specific trained models that each pre-process collected marker information in parallel to generate input features that are processed more efficiently by the second model as compared to known prediction models which distinguish undifferentiated data and thus must be trained with substantially larger training datasets.

### 5.3.3 Genomic and Proteomic Imputation

Genomic imputation, for example DNA Polymorphism (e.g., single nucleotide polymorphism (SNP)) imputation, is typically done for the whole genome at once without prioritizing specific regions in existing imputation methods. In contrast, imputation methods of the technologies are targeted to specific genetic and proteomic markers, i.e., genetic and proteomic information that has been discovered to be statistically significant for predicting treatment outcomes, generating probabilistic outcome measures, and for recommending treatments for specific diseases or immunological conditions, for example for specific cancer types, autoimmune diseases, or other pathologies.

Some exemplary configurations of the technologies described herein use an imputation method that is based on an LSTM autoencoder tuned to perform imputation on regions of interest that are prioritized by relevance to selected conditions or diseases of therapeutic interest. Other imputation methods may also be used as described below. Data mined from one or more data sources, such as the whole genome sequencing data of the thousand genomes project, UK Biobank, and more may be used for this purpose. Further, by leveraging summary statistics from GWASs (both public and private) that are oncology and immunology related, the imputation methods described herein prioritize SNPs that have significant p-values in those GWASs, thereby increasing specificity and accuracy for imputing the prioritized SNPs, including low abundance SNPs, and reducing computing resources by limiting the scope of imputation.

One exemplary aspect of the system is the imputation of missing SNPs for the construction of a shallow long short-term memory (LSTM) autoencoder for four inner layers (e.g., 6 layers overall). The input layer comprises the SNPs identified by particular assay arrays (e.g., from Illumina’s MEGA array, or other arrays) and the output layer provides a complete genome of a target set of genes, for example the complete genome of the set of HLA-A and HLA-B genes. The four inner layers comprise two RNN layers and two convolution layers.

The various layers have selected weights (loss functions) selected as follows.

A set of SNPs (e.g., HLA SNPs) that are not part of the selected assay arrays, and weights associated with each SNP.

Parameters from polygenic risk scores associated with auto-immune diseases.

Parameters from polygenic risk scores associated with cancer pre-disposition.

Parameters identified in the literature (e.g., Stanford GWAS, literature searches).

Similar layer weights and constructions may be used in differing auto-encoder configurations, including parameters directed to proteomic data, or to a combination of proteomic and genomic data.

In a first exemplary embodiment, trained models (1310a, 1301b, 1310c) each include a trained LSTM autoencoder. In a particular exemplary embodiment, each LSTM autoencoder is trained to impute genetic and proteomic information corresponding to a particular disease state, immune state, or condition. For example, a first trained model (1310a) is trained using one or more GWASs corresponding to pancreatic cancer and second trained model (1310b) is trained using GWASs corresponding to breast cancer. When trained in this way, the trained models may impute markers differently due to changes the underlying disease or changes in the disease state, immune state, or condition, which improve the overall accuracy of the results. Similar training may be performed using the corresponding databases for proteomic and mixed proteomic/genetic information. When performing imputation, the system selects one or more of trained models (1310a, 1310b, and 1310c) from the imputation model database (1300), for use in imputing genetic and proteomic information that corresponds to a particular disease or condition of interest. In this manner, the system saves computing resources by only imputing genetic and proteomic information that is associated with the disease or conditions of interest and increases accuracy of the imputations performed as compared to existing imputation methods.

In a second exemplary embodiment, the system includes one or more imputation possibilities databases (2600), each with multiple imputation rule entries. The imputation possibilities database(s) may be stored in a single database, or alternatively, may be stored in multiple databases to segregate imputation rules created for use by a specific source and/or for a specific purpose, e.g., for use with a specific diagnosis. In an exemplary embodiment, at least some of the multiple entries of the database are generated from or by one or more trained machine learning models or are derived from external study types and datasets listed above. In an alternative embodiment, one or more of the entries in the database(s) are generated and entered into the database externally to the system.

In a third exemplary embodiment, the system uses the imputation possibilities database as described above, along with one or more trained models in a two-pass imputation process. The collected data is pre-processed by the system using the imputation database, and then processed a second time using one or more trained models.

Each entry in an imputation possibilities database includes at least one measured genetic or proteomic trait, for example a measured SNP, one or more imputation possibilities corresponding to the measured trait, and, in some embodiments, one or more rules for selecting an imputation possibility for inclusion with imputed markers, for example, imputed genetic information.

The system creates, updates, adds, and deletes rules entries to the imputation possibilities database based on newly processed patient EHR information during a training process. In this manner, the system continuously improves the accuracy of produced imputations as the machine learning models are trained. The continued improvement of entries in the imputation possibilities data allows the imputation possibilities database to improve the imputations recommended by the database as the system continues to process patient data. The improvements in the imputation possibilities database permit further improvement to the imputation results of subsequently processed data, and when used as a collected data pre-processing step, improve the results from the trained models.

An exemplary imputation rule is shown in three-part form,
an evaluation expression specification used to determine the presence or absence of conditions necessary for imputation,
an imputation action, and
a set of imputations.

The evaluation expression specification is a Boolean expression that is evaluated by the imputation rule execution function (part of the imputation program) against a collected dataset and/or processed collected dataset in order to determine a true/false result on whether the imputation action is to be selected and applied to modify the resulting processed collected dataset. The expression is evaluated; if true, imputation is selected and applied, if false, imputation is not performed. Any expression language may be used for the specification of the expression, as long as arbitrarily large sets may be specified.

Expression example syntax

An example expression definition may be encoded using a grammar, such as the example below in Backus normal form (BNF):

Expr ::= { NOT } <gene ID> {<relop> <value>} { ANDIOR <expr>) +

Relop := ∼ | < | = | > | >= | <=

Value := <number, magnitude of expression> | in <gene class/subclass ID>

A non-exhaustive exemplary list of Gene ID’s usable by the embodiments herein is included in Table 1.

Gene class/subclass ID’s are understood in the art. The IN operator treats the class/subclass as a set, and tests for a particular gene being a member of that set.

Table 1

| | | | | | |
|---|---|---|---|---|---|
| Gene ID | Gene ID | Gene ID | Gene ID | Gene ID | Gene ID |
| CXCR5 | KLRF1 | TBX21 | MS4A1 | VPREB3 | GATA3 |
| ASB2 | GNLY | IL12RB2 | CD79A | PAX5 | IL4 |
| CD200 | LILRB1 | TRBV25-1 | CD79B | LILRA4 | IL5 |
| BCL6 | CCL4 | TRAV10 | HLA-DOB | IL3RA | IL13 |
| PDCD1 | NKG7 | ZBTB16 | BANK1 | CLEC4C | IL17RB |
| CD4 | FCGR3A | CD33 | JCHAIN | LAMPS | CCR4 |
| IL17A | KLRD1 | CD14 | IGHM | PTCRA | IL9R |
| IL17F | CD244 | TGFB1 | IGKC | TNFRSF21 | TNFSF10 |
| IL22 | SLAMF7 | CD3D | IGHA1 | FUT7 | IL11 RA |
| KIT | PRF1 | CD3G | IGHG2 | ITGA2B | TNFRSF9 |
| IL17RE | F2R | CD3E | IGHD | ITGB3 | TIGIT |
| RORC | KLRK1 | TRAC | CR2 | CXCL5 | ICOS |
| CTSH | CTSW | TRBC2 | TNFRSF17 | S100A8 | IL2RA |
| LGALS3 | CCL5 | TRBC1 | TNFRSF13B | LYZ | TOX |
| CCR6 | CST7 | IL7R | TNFRSF13C | S100A12 | CCR10 |
| TNFSF13B | TGFBR3 | CD2 | BLK | FCN1 | CCL27 |
| TNFRSF18 | CD300A | TCF7 | FCRLA | MNDA | CCR8 |
| IL1R1 | IL5RA | LEF1 | CD22 | CTSS | CTLA4 |
| CX3CR1 | GZMA | CD27 | FCER2 | MS4A6A | DUSP4 |
| ZEB2 | IL18RAP | CD8A | PDLIM1 | CST3 | FOXP3 |
| ITGAM | KLRG1 | CD8B | POU2AF1 | CSTA | IKZF2 |
| EOMES | GZMK | TRGV2 | TCL1A | CYBB | LRRC32 |
| FCRL6 | CXCR3 | IL32 | CD40 | NCF2 | IL2 |
| GZMH | CCR5 | CD6 | AFF3 | AIF1 | IL20RA |
| GZMB | IFI27 | CD19 | BLNK | CFD | IL21 |
| ITGB1 | SMAD3 | FOXJ1 | ILDR1 | IL37 | HLA-DQB2 |
| CCR7 | IL2RB | IGHG4 | TRAV1-2 | NR1l2 | LAYN |
| SELL | KLRB1 | IGHG1 | CCR1 | CSF2 | TNFSF15 |
| FCER1G | MAP3K1 | IGHA2 | LTK | LIF | CCL7 |
| FCGBP | KIR2DL3 | XBP1 | IL23R | XCL1 | CST6 |
| ENTPD1 | KLRC3 | IGHJ2 | FLT4 | TNFSF11 | CCL13 |
| HAVCR2 | KLRC2 | CD38 | IL4l1 | TXK | CCL8 |
| CXCL13 | KIR3DL1 | IGLG1 | CXCR6 | CD7 | CXCL1 1 |
| LAG3 | KIR2DL4 | IGHJ1 | NCR3 | CRTAM | CLEC4D |
| PRDM1 | KIR2DL1 | TCL1B | CCL20 | FCER1A | CCL2 |
| IL6ST | CD160 | IL21R | CCR2 | ENHO | CD93 |
| TNFRSF10D | EGR1 | IL4R | TRGC1 | CD1C | CLEC4E |
| IRF4 | KIR3DL2 | TGFA | CCL4L2 | CD1E | FCGR1A |
| FAS | CD63 | TRDV2 | PDGFRB | IDO1 | FCGR1B |
| CD58 | ITGA1 | TRDV3 | FCGP2A | CD200R1 | CCL19 |
| SLAMF1 | CCR9 | TRDC | XCL2 | FLT3 | CCL24 |
| NCR1 | CXCR1 | KLRC1 | TRGV4 | XCR1 | CCL26 |
| BACH2 | NCAM1 | ITGAX | FCGR2B | HLA-DQA1 | CXCL12 |
| CD9 | CCL3 | TRGV9 | LYN | HLA-DQB1 | C1QA |
| CD70 | CXCR2 | C1QC | TRGC2 | CD1B | C1QB |
| EBI3 | IL7 | MSR1 | CLEC4F | CD109 | MS4A7 |
| CD80 | FCGR3B | ICAM4 | FCRL1 | CXCL14 | C3 |
| CD44 | FGFBP2 | LTB | ITGAL | HLA-DRB1 | GPR183 |
| ADGRG1 | | | | | |

Examples of evaluation expression specifications include the following. The expression is evaluated; if true, imputation is selected and applied, if false, imputation is not performed.

* Presence of a genetic or proteomic marker or set of genetic or proteomic markers, e.g., {M} or {M, M’ }.

* Presence of a set of genetic or proteomic markers { M, M’ } and the absence of a genetic or proteomic marker { M }.

* Presence of a genetic or proteomic marker with an expression level above a specified threshold (e.g., { [ M, > threshold value ] }, where [M, threshold value] represents M the genetic or proteomic marker, and threshold value is a measured value of the expression of M).

* Presence of a genetic or proteomic marker with an expression level below a specified threshold (e.g., { [ M, < threshold value ] }, where [M, threshold value] represents M the genetic or proteomic marker, and threshold value is a measured value of the expression of M).

* Presence of a genetic or proteomic marker with an expression level equal to a specified threshold (e.g., { [ M, = threshold value ] }, where [M, threshold value] represents M the genetic or proteomic marker, and threshold value is a measured value of the expression of M).

* Presence of a genetic or proteomic marker with an expression level between two specified thresholds (e.g., { [ M, >= threshold 1, <= threshold 2 ] }).

Other expression operators may be added without departing from the scope of the present technology.

The imputation action is a code that indicates how the imputation process should process the information, selected from a list including:

Add - Add the imputations/outcomes data specified to the resulting processed collected dataset.

Remove - Remove the imputations/outcomes data specified from the resulting processed collected dataset (if present)

Modify - Modify existing data in the processed collected dataset in accordance with the imputations/outcomes data.

Modify-relative - Modify existing data in the processed collected dataset in accordance with the imputations/outcomes data by adjusting the reported expression level values (but not changing the reported genetic or proteomic markers) as a function of one or more marker expression levels. For example, increment/decrement an expression level, or set an expression level to a percentage of another gene’s expression level.

Other actions may be added to the system within the scope of the technology.

Exemplary imputation rules include:

- Given an evaluated Boolean result value of “true” for expression X, where X represents an expression as described above, impute and add a set of genes {Y’, Y”, Y’’’, ... } with an imputed strength of expression of each gene in the imputed set.

- Given an evaluated Boolean result value of “true” for expression X, impute and add genetic data represented by Y’, with a corresponding imputed strength of expression that is a function of the measured strength of expression of one or more elements used as parameters of X.

- Given an evaluated Boolean result value of “true” for expression X, add a set of markers {e.g., Y’, Y”} and corresponding imputed strengths of expression.

- Given an evaluated Boolean result value of “true” for expression X, where X includes the testing measured genes X’ and X”, add a set of markers { Y’, Y”, Y’’’ } with specified levels of expression.

- Given an evaluated Boolean result value of “true” for expression X, where X includes the testing of measured gene X’ with an expression level of less than a specified value, modify the specified expression level of X’ to a specified value.

- Given an evaluated Boolean result value of “true” for expression X, where X includes the testing of measured gene X’ with a measured expression level of less than a specified value, modify-relative the specified expression level of X’ by altering the specified expression level of X’ by a function related to the measured expression level (e.g., changing the measured expression level value by a calculated amount or percentage).

- Given an evaluated Boolean result value of “true” for expression X, modify-relative the strength of expression of a gene X’ by setting the strength of expression of X’ as a function of the strength or certainty of an imputation link between an element of X and X’ (e.g., based upon the strength of linkage of SNPs to a particular disease or disorder).

- Given an evaluated Boolean result value of “true” for expression X, impute and add a set of markers {Y’, Y”, Y”’} and based upon the imputed marker Y’, further impute marker Z’, which is related to Y’, to produce the set of imputed markers {Y’, Y’’, Y’’’, Z’}.

- Given an evaluated Boolean result value of “true” for expression X, impute by deleting the set of markers {Y’, Y’’, Y’’’}.

Further exemplary rules can specify imputation strength scores as a function of one or more additional or alternative factors that may influence imputation accuracy including, for example, SNP density and sequencing depth or coverage.

In a first exemplary embodiment, the system uses the marker information extracted from the collected dataset to marker(s) identified in one or more instances of imputation possibilities database(s) (2600) and uses the associated rules to determine imputed marker data based on the measured marker(s) and RX/DX information in the collected dataset.

In a second exemplary embodiment, one or more of the trained models (1310a, 1310b, and 1310c) compares marker information extracted from the collected dataset to markers identified in one or more instances of genetic and proteomic possibilities database(s) (2600) and uses the associated rules to determine imputed markers based on the measured markers in the collected data. In an exemplary embodiment, the trained model(s) can function as expert systems modules by identifying and implementing only those rules contained in the imputation possibilities database necessary to produce imputed markers based on the observed markers (e.g., gene IDs such as those examples enumerated in Table 1) and a newly identified RX or DX value generated by the trained model. In this manner, the system can generate imputation results using fewer computing resources as compared to traditional imputation methods and programs.

In some exemplary embodiments, the system uses one or more additional or alternative methods to impute markers based on measured markers including, for example, Bayesian approaches and graphical causal models.

### 5.3.4 Low Expressed Genetic and Proteomic Information

Some exemplary genetic and proteomic markers include low expressed markers, for example low abundance markers, e.g., rare SNPs or other rare variants. “Low expressed genetic information” is defined herein as genetic information which may be difficult to characterize by a default or usual sequencing depth or coverage used by the system. “Low expressed proteomic data” is defined herein as proteomic information which may be difficult to characterize by a default or usual proteomic analysis techniques. Low expressed genetic and proteomic information may also be difficult to characterize by imputation, for example due to low levels of association or linkage with other markers that are more readily observed. In an exemplary embodiment, the system parses a patient EHR and determines genetic and/or proteomic markers contained therein, for example, markers determined by a low coverage or low depth, e.g., 4X or 8X, sequencing performed on one or more biological samples (blood cells/PBMCs, tumor cells, etc.) from the patient. The system determines whether markers are missing from the initial sequencing results, i.e., from the observed markers, and, if so, uses one or more of the imputation methods of the exemplary technology described herein to attempt to impute the missing markers, which can include low abundance markers. Because the imputation methods are designed to impute specific markers, as previously described, low abundance markers are more readily and accurately identified by the imputation methods.

If, however, the system determines that one or more low abundance markers have not been observed and additional marker information has not been successfully imputed, the system can recommend (using a recommender program like the low abundance method recommender program FIG. 4, 3750) further tests to characterize the low abundance markers, for example by performing deeper and/or targeted sequencing on the one or more biological samples that were previously studied or from one or more biological samples newly acquired from the patient. In a particular embodiment, the recommender system includes a database of methods for generating additional genetic and/or proteomic data. In an exemplary embodiment, the database of methods for generating genetic and/or proteomic data includes, for example, one or more of amplification methods, sequencing workflows, and targeted assays that may be recommended as defined in the recommended tests database (3775). The system selects from the database one or more methods for generating genetic and proteomic data specific to the one or more missing or low abundance markers. For example, the system selects an amplification method targeted for a particular low abundance genetic marker in order to obtain new data to complete the collected data. The system then recommends performing the selected methods/procedures for generating genetic data on one or biological samples from the patient.

### 5.3.5 Exemplary System Architecture

FIG. 2 illustrates an exemplary computer system (2000) of standard manufacture that provides and implements one embodiment of the training aspects of the described system. An exemplary computer system comprises one or more processors (2210), persistent and transient memories for storing data and programs (collectively 2220), storage access circuitry that enables the processor to read and write data and programs from and to the memories, and one or more network or other communications interface(s) (collectively 2230) (e.g., an Ethernet, 802.11, cellular radio transceiver, and direct hardware interface operably connected to external databases comprising training datasets and trained model databases). An exemplary computer system further comprises, in at least one of its persistent memories, one or more programs (collectively 2200), which may include specialized programs to implement collected dataset processing (e.g., extractor/encoder 2250), genetic and proteomic information imputation (e.g., imputation program 2650), and trained model and rule generation (e.g., model/rule generator 2260) as described herein.

EHR extractor/encoder program (2250) receives, from collected data database (1100), historical patient EHRs, e.g., EHRs from a cohort of patients comprising a particular patient population of interest, and mines the patient EHRs datasets in order to generate datasets including DX, RX, outcomes, and genetic and proteomic data associated with the EHRs data, which is stored in training dataset storage database (2275).

The imputation program (2650) receives, from training dataset storage database (2275), collected data including genetic and proteomic data and uses one or more imputation methods to produce processed collected data that includes imputed genetic and proteomic marker information. In an exemplary embodiment the imputation program produces markers associated with those genetic and proteomic markers identified for imputation, as defined herein. The imputation program stores processed collected data, including imputed genetic and proteomic marker information, in the training data storage database.

Model/rule generator program (2260) receives, from training dataset storage database (2275), collected and/or processed collected data including genetic and proteomic information and other patient-related data (e.g., DX, RX, outcomes). In a first exemplary embodiment, the model/rule generator program receives, from model storage database (2270) one or more untrained models. The model/rule generator program uses the collected and/or processed collected data to train the untrained models, thereby generating trained imputation models (e.g., 1310a, 1310b, 1310c) which it stores in the external trained model database (2300). In a second exemplary embodiment, the model/rule generator program uses the collected and/or processed collected data to generate one or more imputation rules, which it stores in imputation possibilities database (2600).

The computer system may optionally further comprise one or more database((s)/cache(s) (e.g., databases 2270, 2275) in which the computer stores information about one or more of collected data, processed collected data, training datasets, and trained models with which it is configured to inter-operate. The database/cache may be stored in an internal memory of the computer system (e.g., model storage database 2270, training dataset storage database 2275) or may be stored in an external database such as the collected dataset database (1100), the trained model database (2300), or the imputation possibilities database (2600) to which the system is operably connected. In some embodiments, the computer system may further comprise a secure key storage mechanism (not shown) such as a secure processor, a TPC chip, or other mechanism in which it stores cryptographic materials that protect patient confidential information.

When operating, the exemplary computer processor executes one or more of the exemplary systems’ programs from persistent or transient memory in order to convert the general purpose computer into a processor-controlled role-specific device that implements the one or more defined processes of the executing programs.

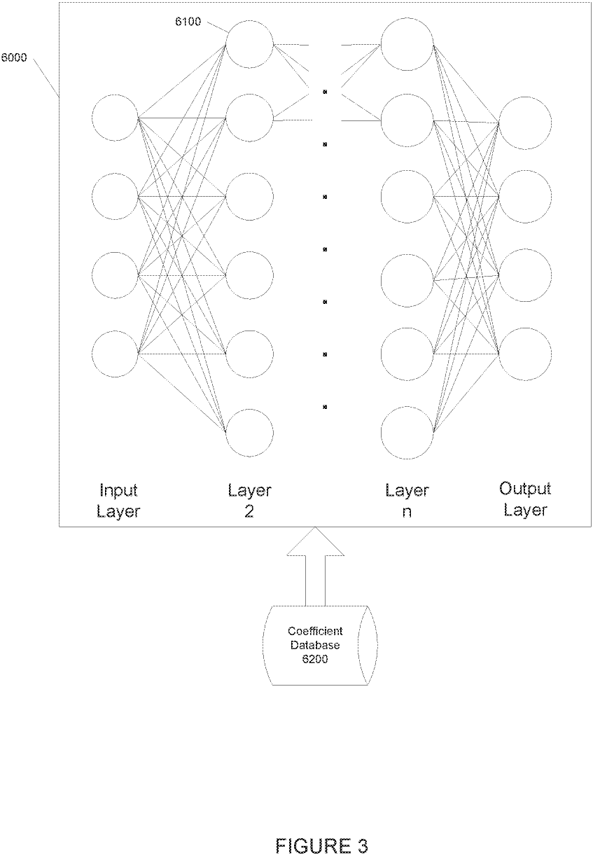

FIG. 3 illustrates an exemplary deep neural network engine (6000) using configurable coefficient storage in the form of a coefficient database (6200). When combined within an exemplary computer system (e.g., system 3000 described below), the neural network engine is configured by the processor by selecting coefficients (6100) read from a coefficient database and populating the neural network with those coefficients. Exemplary coefficients comprise parameters including weights and biases associated with individual neurons or blocks of a particular neural network configuration and in some embodiments include specification of one or more of activation functions and transfer functions. For clarity, the drawing omits most of the inter-neuron relationships. The neural network engine may be implemented as stand-alone computer hardware system, or as a module within a larger computer system without deviating from the scope of the present technology. The neural network engine may include neural network configurations of differing types. In an exemplary embodiment, the neural network engine includes a feed forward neural network, for example a feed forward back propagation neural network. In further embodiments the network engine includes one or more additional or alternative neural network configurations, alone or in combination, including, but not limited to, recurrent neural networks (RNN), for example long short-term memory (LSTM) RNN, convolutional neural network (CNN), Bayesian or Belief neural network (BNN), or another directed acyclic graph configuration of a neural network. In some exemplary embodiments, the neural network engine includes a neural network configuration having multiple hidden layers of differing types, for example one or more RNN layers and one or more CNN layers.

FIG. 4 illustrates an exemplary computer system (3000) of standard manufacture that provides and implements one embodiment of the predictive aspects of the described system. An exemplary computer system comprises one or more processors (3210), persistent and transient memories for storing data (collectively 3220), storage access circuitry that enables the processor to read and write data and programs from and to the memories, and one or more network or other communications interface(s) (collectively 3230) (e.g., an Ethernet, 802.11, cellular radio transceiver, and direct hardware interface operably connected to external databases comprising patient EHR data and trained model databases). An exemplary computer system further comprises, in at least one of its persistent memories, one or more programs (collectively 3200), which may include specialized programs to implement dataset collection and extraction (e.g., patient data/extractor program 3280)), imputation (e.g., genetic and proteomic imputation program 3650, a set of recommender programs (e.g., low abundance method recommender program 3750), and treatment/outcome prediction (e.g., prediction program 3290) as described herein.

Patient data extractor program (3280) receives, from patient data database (1500), collected data corresponding to a selected patient. In some embodiments (not shown), the patient data extractor receives data directly from an EHR, and/or directly from a genetic reader. When new data is obtained, the patient data extractor program writes the collected data to the patient data database (1500). The patient data extractor program then mines the patient’s collected dataset to identify aspects of the collected data including DX, RX, outcomes, and collected genetic and/or proteomic data. In some embodiments, the patient data extractor program receives additional information identifying selected markers (and/or disease profiles) from a markers and statistics database (1120) and limits the collected data to include only those identified markers. In additional exemplary embodiments, the patient data extractor program encodes the collected data for use by the system, for example, by standardizing aspects of the collected data by converting numerical laboratory test results to standardized ranges (e.g., low, normal, high). The patient data extractor program stores the collected data in system database (3270) or back to the patient data database (1500). In some embodiments, the patient data extractor program provides the collected genetic and proteomic data directly to the genetic and proteomic imputation program (3650).

Genetic and proteomic imputation program (3650) receives collected information from either the patient data extractor program (3208), system database (3270), or both and produces imputed marker(s) of one or more genetic and proteomic data in the dataset as processed collected data, for example, creating specific (genetic and/or proteomic) markers (some or all of ID, values, and/or expression levels), that are missing from the collected data. In a first exemplary embodiment, the imputation program retrieves, from external trained model database (2300), a trained imputation model and uses the trained imputation model to produce the imputed markers. In a second exemplary embodiment, the imputation program retrieves, from imputation possibilities database (2600) one or more imputation rules and uses the one or more imputation rules to produce the imputed genetic and proteomic information. The imputation program stores the processed collected data, including imputed information, in system database (3270) or in the patient data database (1500). In a third exemplary embodiment, the imputation program selects and applies imputation rules to pre-process the collected data, and then selects and uses the trained imputation model to further process the collected data.

In some exemplary embodiments, the low abundance recommender program (3750) retrieves, from system database (3270) or the patient data database (1500), collected and processed collected data and determines genetic and proteomic information including expression levels of one or more retrieved markers that require imputation to create, modify, or removes information from either the collected and processed collected data. In some implementations, the system automatically selects one or more imputation steps to be performed. In other cases, the low abundance recommender program (3750) generates one or more recommendations for additional tests that may be run to create the missing markers and stores these recommendations in recommended tests database (3775). In some implementations, the recommendations may be produced in a form of a prescription that is subsequently made available to a testing provider, or is presented on an output device.

Prediction program (3290) receives, from system database (3270), one or more of collected and processed collected data and receives, from external trained model database (2300), one or more trained prediction models. The prediction program (3290) uses the one or more trained prediction models to generate, based on the collected and processed collected data, one or more predicted outcomes including, for example, DX, RX, outcomes, and projected survival duration. The prediction program (3290) stores the predictions in prediction database (3600).

In some embodiments, the prediction program compares successively generated stored predictions to subsequent predictions and outcomes to identify those cases where earlier predictions were not accurate. Those prediction cases where the prediction is less accurate than a specified threshold (stored in a configuration) and the underlying patient dataset may be copied to a collected data database (1100) for use in producing new iterations of trained models.

The computer system may optionally further comprise a local instance of one or more system database/cache (3270) in which the computer stores information about one or more patient data and trained models with which it is configured to inter-operate. The database/cache (3270) may be stored in an internal memory of the computer system (not shown) or may be stored in an external database such as the patient database (1500), the markers and statistics database (1120), the trained model database (2300), the prediction database (3600), the recommended tests database (3775), and the imputation possibilities database (2600) to which the system is operably connected. In some embodiments, the computer system may further comprise a secure key storage mechanism (not shown) such as a secure processor, a TPC chip, or other mechanism in which it stores cryptographic materials that protect patient confidential information.

When operating, the exemplary computer processor executes one or more of the exemplary systems’ programs from persistent or transient memory in order to convert the general purpose computer into a processor-controlled role-specific device that implements the one or more defined processes of the executing programs.

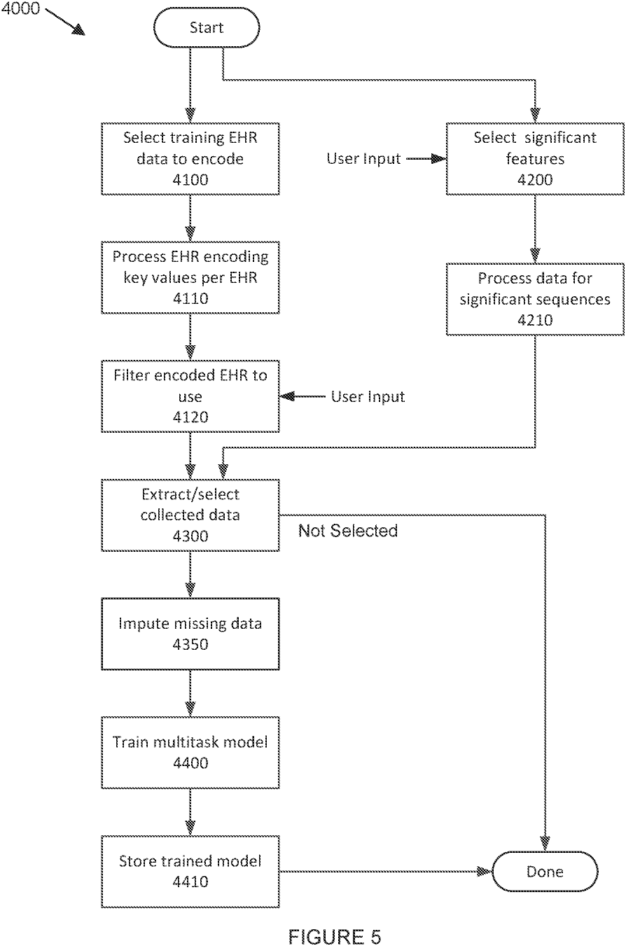

FIG. 5 illustrates an exemplary process (4000) performed by the programs of the training computer system for processing genetic data. A similar process is performed when processing protein and proteomic data, and for processing mixed genetic and protein and proteomic data. The training system has two paths, one for training the multitask model and one for establishing the database of prioritized features (per the aforementioned GWAS and other private studies) used by the system. On the path for establishing the database of features, in step 4200, the user first selects significant features for a specific DX from the known literature and databases of summary statistics information. In step 4210, the system then processes the data in order to extract significant genetic sequences and associate these sequences with the DX and statistical significance of the sequences. The processed information is then provided to the training system for training the multitask model, and may be optionally stored in a database (step not shown) for later reuse.

On the path for training the multi-task model, in step 4100, the system selects one or more training datasets from either the collected data database (1100) or a training data storage database (2275) to encode/process. The selection may be on the basis of one or more identified DX, RX, outcomes, or on the basis of other factors determined by the user. In step 4110, the training dataset(s) are then processed, extracting and encoding the features needed to train a predictive model according to the technologies described herein. The extraction and encoding may include DX, RX, outcome, and marker extraction and encoding.

In step 4120, the resulting training dataset is then filtered based upon user input to exclude anomalous data, and the resulting filtered training dataset is combined with the processed statistical significance information from step 4210 to produce a combined training dataset.

In step 4300, the combined training dataset is then down-selected on the basis of the identified information present. The down-selection process identifies the information in the combined training dataset that will be useful in training a multi-task model for a selected DX/RX/genetic pair. If no data is selected, the current training session ends.

If there are training dataset(s) associated with to the specific training activity, the system selects those training dataset(s) and proceeds to step 4350 where it performs imputation steps to produce imputed markers from among the genetic information identified in step 4300 as useful for training the multi-task model. The system performs the imputation using previously generated imputation trained models. Alternatively, the system may select an imputation possibilities dataset which may select and use imputation rules from the selected dataset in the imputation process.

The system then proceeds to step 4400, where it creates a trained model for the specific DX/RX/marker using the selected elements of the combined training dataset. In step 4402, the system determines whether the multitask model has been optimized, for example by generating predictions using the model and labeled testing data, calculating a cost function based on comparison of the generated predictions and the testing data labels, and determining whether the cost function has been minimized. If the system determines that the model has not been optimized, in some embodiments it proceeds to step 4405 to update imputation models and/or imputation rules. The system then repeats imputation step 4350 and to produce new imputation results performs additional training on the multitask model using training data including the new imputation results.

If the system determines, at step 4402, that the multitask model has been optimized, it proceeds to step 4410 and the resulting trained model is then stored in a database such as the trained model database (2300), following which the training process completes. The training process may be repeated as often as necessary as new training dataset(s) or useful statistical significance data is made available, or when a new trained model is desired for a different DX/RX/genetic pair.

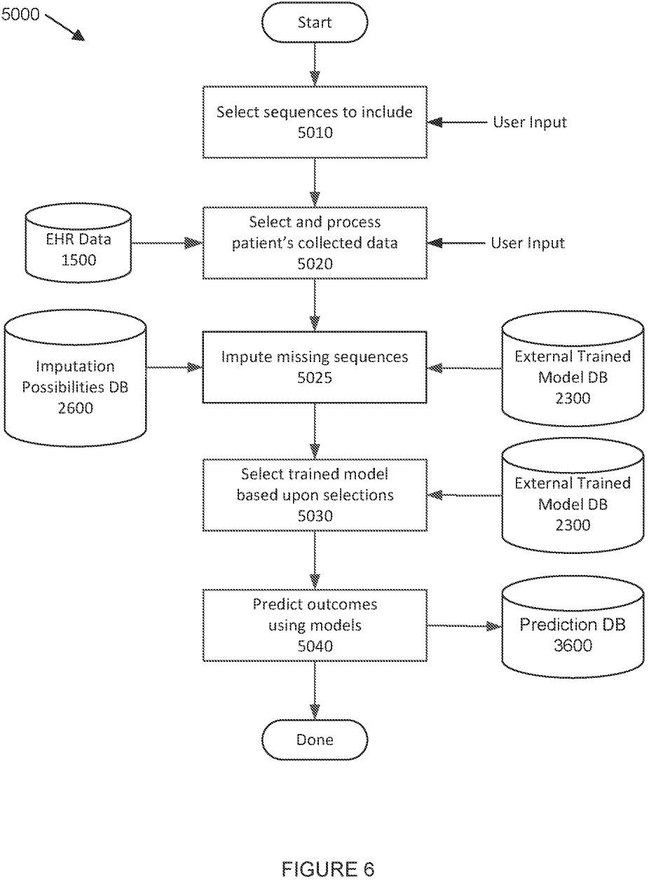

FIG. 6 illustrates an exemplary process (5000) performed by the programs of the predictive computer system.

The process starts with step 5010, where the user selects genetic sequences to include in the prediction.

In step 5020, the system processes the patient’s collected dataset from the patient database (1500), selecting and extracting, on the basis of user input, the patient’s DX, RX, treatment stage/outcome(s) to date, one or more collected datasets (e.g., VCFs), and, in some exemplary embodiments, microbiome features, e.g., alpha or beta diversity). User inputs may include the following:

User selection of results as absolute or relative survival curves.

User selection prioritizing or omitting different genetic features.

User selection prioritizing or omitting different proteomic features.

User option to manually enter clinical information (i.e., single gene mutations).

At step 5025, the system performs any imputation necessary (1650 of FIG. 1) to adjust the collected dataset using one or more of the imputation trained models (e.g., 1310a, 1310b of FIG. 1). Imputation is used to enhance the patient’s collected dataset (e.g., EHR data or directly read genetic data) by adding imputed markers for missing information, thereby producing a processed collected dataset (e.g., 1675 of FIG. 1).

This permits the user to have control over the types of information used by the trained models. The selected information is then used to select one or more trained models (e.g., 1410a, 1410b of FIG. 1) from the trained model database (2300) in step 5030.

In step 5040, the selected trained model(s) are used to predict the outcomes based upon specific treatments and based upon at least some of collected data and/or processed collected data, including collected and, in some cases, imputed markers (1700 of FIG. 1).

In a particular exemplary embodiment, the system uses one or more selected trained models to generate multiple predicted outcomes, each corresponding to a specific treatment, thereby generating a set of treatment and predicted outcome pairs. The system further uses the trained models to generate a likelihood or probability metric associated with each of the predicted outcome/treatment pair and generates a ranked list comprising treatment/predicted outcome pairs, ordered according to the predicted outcomes and their probability metrics. The system then generates one or more treatment recommendations based on the ranked list. In an exemplary use case, the system uses the selected trained models to generate, for a particular cancer, predicted outcomes of up to four different treatments and a probability metric associated with each predicted outcome.

The selected trained models also operate as a recommender engine and produce one or more recommended treatments, in addition to predicting outcome, survival, and disease progression milestones and disease progression timelines. In an exemplary embodiment, the system uses the predicted outcomes to generate one or more treatment recommendations. The system may filter the predicted outcomes to eliminate any predicted poor outcomes, for example patient death or non-response. In one exemplary embodiment, the one or more treatment recommendations may include each treatment that was not associated with a poor outcome. In another exemplary embodiment, the one or more treatment recommendations include recommendations selected based on one or more additional criteria, for example treatments associated with a probability metric have a value greater than a threshold value or are the top treatments on a list of treatments ranked by probability metric values, e.g., the top two treatments or the top three treatments. These outcome predictions and treatment recommendations (combined model 1415 of FIG. 1) are stored in prediction database (3600) for subsequent use and comparison if the patient is re-processed using the system, for example the patient’s EHR is updated with newly collected data at a subsequent time point.

In an exemplary embodiment, when newly collected patient outcome data is added to a patient’s EHR, the system retrieves from the prediction database any predicted outcomes previously generated by the system and compares the newly collected outcome data to the previously predicted outcomes. If the newly collected outcome data does not agree with the previously predicted outcomes, the system may retrain the model(s) used to generate the outcome(s) or use one or more known reinforcement learning methods to update the trained models.

The system may also generate recommendations for additional testing to be performed. For example, the system may recommend additional testing to generate missing markers of low abundance data that it is unable to generate using imputation methods of the technology, as described herein.

If the system produced one or more recommended treatments and/or recommended additional testing, a user of the system may take actions based upon the recommendation to convert one or more recommendations into a prescription/testing order for the identified additional tests and/or treatments. Referring back to FIG. 1, the system provides for an optional feedback loop in which the stored outcome predictions are compared against one or more subsequent patient state information (e.g., updated RX, DX information) and the accuracy of the prediction assessed. The prediction accuracy may be used to increase and decrease internal weightings in the predictive models used to make the predictions.

Similarly, by using RX and DX data compared against the outcome predictions, the system may provide an assessment of the current progression of the patient’s disease in relation to the predicted disease progression predictions. Updated predictions may also be made on the basis of this assessment.

### 5.4 Example 1. Imputation of Missing Genomic Data

The system collects multi-omic (germline sequence data, mutation data, single cell RNA sequence (scRNAseq) data, microbiome sequence data, metabolomic data, epigenomic, and genetic and genomic test results) single cell data Single cell data is often quite sparse, in some cases because of frequent “dropout events”, meaning that the sequence of a gene that is expressed even at a relatively high level may not be detected because of technical limitations of the assay, such as, for example, the relative inefficiency of reverse transcription. This type of error can lead to significant problems with cell-type identification with the machine learning and other downstream analyses. Completion and improvement of the collected data set provides improved machine learning (and downstream analysis) results.

Table EX1 illustrates the input and output results of imputing the expression of a particular gene based upon the expression of other genes obtained through single cell RNA sequencing (scRNAseq) per cell.

| INPUT (pre imputation) | | Cells | | | |
|---|---|---|---|---|---|
| scRNAseq data | | CD8 EM | CD9 memory | CD8 CM | CD8 temra |
| | PDCD1 | High | High | Low | Medium |
| | GZMB | Medium | Low | Low | High |
| | ZEB2 | High | Low | High | Low |
| | LAG3 | (no data) | (no data) | (no data) | (no data) |
| OUTPUT (post imputation) | | Cells | | | |
| scRNAseq data | | CD8 EM | CD9 memory | CD8 CM | CD8 temra |
| | PDCD1 | High | High | Low | Medium |
| | GZMB | Medium | Low | Low | High |
| | ZEB2 | High | Low | High | Low |
| | LAG3 | HIGH | LOW | LOW | MEDIUM |

### 5.5 Conclusions

It will also be recognized by those skilled in the art that, while the technology has been described above in terms of exemplary embodiments, it is not limited thereto. Various features and aspects of the above described technology may be used individually, jointly or in combination. Further, although the technology has been described in the context of its implementation in a particular environment(s), and for particular application(s), those skilled in the art will recognize that its usefulness is not limited thereto and that the present technology can be beneficially utilized in any number of environments and implementations where it is desirable to provide a highly accurate treatment recommender and condition monitoring system or other implementations. Accordingly, the claims set forth below should be construed in view of the full breadth and spirit of the technology as disclosed herein.

1. A system for variably imputing genetic and/or proteomic information into a collected data set, comprising:
storage access circuitry structured to read an input genetic and/or proteomic data set comprising genetic and/or proteomic expression data including genetic and/or proteomic test results, and to read at least one imputation data set from a database, the imputation data set comprising one or more imputation technique definitions for variably imputing collected data; and
an imputation engine coupled to a storage access circuitry, the imputation engine applying the one or more imputation technique definitions from the at least one imputation data set to a collected genetic and/or proteomic data set to variably impute genetic and/or proteomic data and create a resulting processed dataset
wherein the storage access circuitry is further configured to output the resulting dataset.

2-55. (canceled)
